(19) United States
(12) Patent Application Publication
(10) Pub. No.: US 2006/0106419 A1
(43) Pub. Date: May 18, 2006

(54) Three dimensional implant

(76) Inventor: Peter Gingras, Galway (IE)

Correspondence Address: FISH & RICHARDSON PC, P.O. BOX 1022, MINNEAPOLIS, MN, 55440-1022, US

(21) Appl. No.: 10/525,193

(22) PCT Filed: Aug. 25, 2003
(86) PCT No.: PCT/US03/26905

§ 371 Date: Aug. 12, 2005

Related U.S. Application Data
(60) Provisional application No. 60/405,517, filed on Aug. 23, 2002.

(51) Int. Cl. A61B 17/08

(52) U.S. Cl. 606/213

(57) ABSTRACT

Implants (20, 22) and methods of making the implants for treating bodily defects or remodeling tissue. The implants have a low density and open pores (49) which may permit tissue ingrowth.

### TECHNICAL FIELD

This invention relates generally to medical devices and more specifically to three-dimensional implants that can be administered to injured or otherwise defective tissue within the body.

### BACKGROUND

Soft tissue implants are commonly used to reinforce or replace areas of the human body that have acquired defects. Several soft tissue implants have been developed and are commercially available. For example, Bard Mesh™ is a non-absorbable implant that is made from monofilament polypropylene fibers using a knitting process (C.R. Bard, Inc., Cranston, R.I.; see also U.S. Pat. No. 3,054,406; U.S. Pat. No. 3,124,136; and Chu et al., J. Bio. Mat. Res. 19:903-916, 1985). This same material is used to construct other implants such as the Bard Mesh PerFix™ Plug, discussed further below.

Soft tissue implants have been used to treat many defects, including those that affect the abdomen and abdominal wall. For example, cylindrical plugs have been suggested for recurrences of inguinal hernia (Lichtenstein et al., Am. J. Surg. 128:439-444, 1974), and an umbrella plug technique was subsequently described (Gilbert, Perspectives in General Surgery 2:113-129, 1991). Yet another technique for mesh plug hernioplasties was described by Rutkow in 1993 (Rutkow et al., Surgery 114:3-8, 1993). Abdominal wall defects can also be addressed with the Bard Mesh PerFix™ Plug, which functions as an implantable and non-absorbable mesh prosthesis that can be used as a compressible and pliable implant (C.R. Bard, Inc., Cranston, R.I.; see also U.S. Pat. Nos. 5,356,432 and 5,716,408; see also U.S. Pat. No. 6,066,776). Implantable prostheses for repairing defects in muscle or other tissues can have a preformed shape that conforms to the shape of the defect. The shaped prosthesis may facilitate placement and minimize shifting (see U.S. Pat. No. 5,954,767). Kits that can be used to repair indirect hernias are described in U.S. Pat. No. 6,166,286, and a prosthetic device having an extension canal made of sheet material for extending through a hernia is described in U.S. Pat. No. 6,241,768. An implantable prosthesis containing a radially-expandable member for placement in and occlusion of a hernia opening is described in U.S. Pat. No. 6,425,924.

The plugs described above are made using synthetic fiber technology. The implant surface area for the biomaterial used to construct the Bard Mesh™ has been calculated. The following formulas were used to calculate the surface area ratio for Bard Mesh:

Vₘₐₜ=Wₘₐₜ/Dₘₐₜ where Vₘₐₜ is the material volume, Wₘₐₜ is the material weight, and Dₘₐₜ is the material density which is 0.904 g/cm³ for polypropylene;

L_{fiber}=Vₘₐₜ/((II)(R_{fiber})²) where R_{fiber} is the radius of the fiber and L_{fiber} is the length of the fiber;

A_{surface}=(II)(D_{fiber})(L_{fiber}) where A_{surface} is the surface area of the fiber used to construct the material and D_{fiber} is the diameter of the fiber; and

Surface Area Ratio=A_{surface}/Fₐᵣₑₐ where Fₐᵣₑₐ is the area of the biomaterial fabric used to obtain Wₘₐₜ.

| | | Weight | Fiber | Surface Area |
|---|---|---|---|---|
| Product | Construction | (g/cm²) | Diameter (cm) | Ratio |
| Bard Mesh | Monofilament | 0.0096 | 0.017 | 2.52 |
| | Knit | | | |

Bard Mesh™ is used to construct the Bard Mesh PerFix™ Plug. With values for the implant surface area for the Bard Mesh™ and the volume for the Bard Mesh PerFix™ Plug, the implant surface area to volume ratio can be calculated. The following formulas were used to calculate the surface area to volume ratio for the PerFix™ Plug:

A_{surface plug}=(W_{plug}/W_{mesh cm2})*A_{surface cm2} where A_{surface plug} is the surface area of the fiber used to construct the plug, W_{plug} is the weight of a size large PerFix Plug, W_{mesh cm2} is the weight of Bard Mesh per cm² and A_{surface cm2} is the area of the fiber used to construct Bard Mesh per cm²;

V_{plug}=((II)(L_{plug})(R_{plug})²)/3 where V_{plug} is the volume of the cone shaped plug, L_{plug} is the plug height, and R_{plug} is the plug radius at the base; and

Surface Area to Volume Ratio=A_{surface plug}/V_{plug}.

| | | Plug Surface | Plug Volume | Surface |
|---|---|---|---|---|
| Product | Weight (g) | Area (cm²) | (cm³) | Area:Volume |
| PerFix Plug | 1.01 | 266 | 24.68 | 10.79 |
| (Large) | | | | |

These implants are not ideal. Following are some of the disadvantages associated with one or more of the implants presently used. Where their construction results in substantial wall thickness, surface area, density, and/or interstices, there is an increased risk of inflammation and infection; loose or soft plug implants can collapse, leading to shrinkage during the healing process (up to 75%, which can fail to secure the intended repair); excessive scarring and shrinkage can cause plug implants to assume a cartilage-like consistency (which can erode into adjacent tissue such as the bladder, intestines, and blood vessels); in the event of neuralgia, plugs may have to be removed; material content and wall thickness can require large incisions (thus, utility in less invasive surgical procedures may be limited); seromas, caused by the host inflammatory reaction to the implant, and dead space can be created between the prosthesis and host tissue; rough implant surfaces can irritate tissues and lead to the erosion of adjacent tissue structures and adhesion to bowel when the implant comes in direct contact with the intestinal tract; non-absorbable implants may elicit a chronic foreign body response; implants having small pores may not permit adequate tissue ingrowth and incorporation; implants requiring a separate onlay require additional time to implant; and plug implants are prone to migration, even with the use of staples or sutures. Accordingly, there remains a need for devices for repairing soft tissue bodily defects.

### SUMMARY OF THE INVENTION

The present invention features implants (e.g., three-dimensional soft tissue implants) that can be used to treat bodily defects (whether arising congenitally or as a result of a disease, disorder, condition, or surgical procedure) or to remodel tissue (following, for example, a traumatic injury (such as a burn) or for cosmetic purposes). In addition, the invention features methods for making the implants and kits (e.g., sterile kits that include an implant and, optionally, instructions for its application, and which can facilitate the surgical procedure in which the implant is used). In one embodiment, the implants have a low density (i.e., a low weight:volume ratio), a low implant surface area ratio (the fiber or material surface area divided by the material area), and open pores, which may permit tissue ingrowth following implantation into a patient (e.g., a human patient). The surface area ratio can range from about 0.4 to about 4.0. For example, the surface area ratio can be approximately 1.0, 2.0, or 3.0 (e.g., 0.6, 0.8, 1.0, 1.2, 1.4, 1.6, 1.8, 1.9, 2.0, 2.1, 2.2, 2.4, 2.6, 2.8, or 3.0; the implants of the invention can also be described as having a surface area ratio of less than 3.0), the surface area to volume ratio can range from about 2.0 to 4.0. For example, the surface area to volume ratio can be approximately 3.0 (e.g., 2.0, 2.1, 2.2, 2.4, 2.6, 2.8, 3.0, 3.1, 3.2, 3.4, 3.6, 3.8, or 4.0), and the pore size can be approximately 50-2000μ (e.g., 50, 100, 200, 300, 400, 500, 600, 700, 800, 900, 1000, 1100, 1200, 1300, 1400, 1500, 1600, 1700, 1800, 1900, or 2000μ; preferably, the pore diameter is measured before implantation, when the implant is in a natural, resting position). Thus, and as further described below, the methods of the invention can produce implants that are highly porous and of a low material content, yet strong enough to modify for repair tissue.

More specifically, the invention features a three-dimensional biocompatible implant, the implant comprising a subassembly that resists compression when implanted in a warm-blooded animal. The subassembly can include woven or braided fibers or can be produced using a circular weft or warp knitting process. In any event, the subassembly can be produced using an internal support (e.g., PEEK). The implant of claim 1, wherein the subassembly is produced using a circular warp knitting process. The subassembly can also be produced using a nonwoven film and/or a substrate comprising pores as shown in FIG. 9C. The pores can be 50-2000 microns in diameter, and the implant can have a conical form and further include an only or anchor.

The invention also features a method for producing a three-dimensional biocompatible implant that includes one or more of the following steps:

a) extruding a biocompatible polymer into a fiber,

b) transforming the fiber into a compression resistant subassembly,

c) braiding or weaving the subassembly into a three dimensional structure,

d) heat setting the structure into the desired shaped article, and, optionally,

e) attaching the shaped article to a complementary implant article.

The invention also features a method for repairing a defective tissue in a patient (e.g., a patient with a hernia), the method comprising applying the three-dimensional biocompatible implant to the defect by way of a surgical procedure.

The invention also features a kit comprising an implant, optionally sterile, as described herein.

The invention also features a method of delivering the implant of claim 1 to a patient's body, the method comprising exposing a defective tissue on or within the patient's body and placing the implant on or over the tissue. The implant can be compressed, by hand or by a device, prior to being placed on or over the tissue.

The method for producing a three-dimensional biocompatible implant, the method comprising one or more of the following steps:

a) extruding a biocompatible polymer into a film,

b) transforming the film into a subassembly,

c) shaping the subassembly into a three dimensional structure,

d) heat setting the structure into the desired shaped article, and, optionally,

e) attaching the shaped article to a complementary implant article.

The invention also features a three-dimensional implant comprising two or more layers of two-dimensional biocompatible material with interconnecting supports, said implant constructed to securely fit within a tissue or muscle wall defect.

The implants of the invention may have one or more of the following advantages. They can be configured to allow or stimulate fibrosis (or fibrotic tissue ingrowth) in an organized pattern (tissue ingrowth under these circumstances may provide additional support to the previously defective tissue); they can have a reduced surface area and/or density (reduced with respect to present implants) that minimizes the inflammatory response and infection risk to the patient; they can have a degree of compression resistance that minimizes shrinkage and erosion of the implant into adjacent tissue structures and reduces the likelihood of collapse after insertion; they can have stress-strain properties that are compatible with the mechanical properties of the tissues they contact in the patient's body and therefore promote healing and minimize discomfort; they can be constrained (e.g., held within a biocompatible (e.g., non-toxic) tube or similar outer structure) and have a profile low enough to facilitate insertion and deployment within a patient in a minimally invasive fashion; they can be biodegradable or bioresorbable; and they can contain an onlay or anchor that can be secured in position in a short period of time. The anchor and three-dimensional soft tissue implant combination create a frictional force with the surrounding tissue to prevent migration. Implants that are less prone to migrate from the site of implantation can be used with fewer, if any, staples or sutures (it is expected that this will reduce complications associated with attachment to the surrounding tissues). Moreover, the implants of the invention can be economical to manufacture and highly reproducible, durable, and efficient.

Still further objects and advantages will become apparent from a consideration of the ensuing description and drawings.

### BRIEF DESCRIPTION OF THE DRAWINGS

FIG. 1 is a perspective view of a weft knit subassembly.

FIG. 2 is a perspective view of a warp knit subassembly.

FIG. 2A is a perspective view of a weft knit subassembly with an internal support.

FIG. 3 is a perspective view of an implant.

FIG. 4 is a perspective view of a shaped implant.

FIGS. 5A-5C are perspective views of implants in various configurations. FIG. 5A illustrates an implant in a collapsed constrained configuration; FIG. 5B illustrates an implant in a collapsed constrained configuration positioned within a bodily defect; and FIG. 5C illustrates an implant in an unconstrained configuration within a bodily defect.

FIG. 6A is a perspective view of a three-dimensional implant with connection means to other implants.

FIG. 6B is a perspective view of the implants connected closely together.

FIG. 6C is a cross-sectional view of the connected implants positioned within a bodily defect.

FIG. 7 is a diagram showing the manufacturing steps.

FIGS. 8A-8C are perspective views of implants in various configurations. FIG. 8A is a perspective view of a non-woven subassembly; FIG. 8B is a non-woven three dimensional implant; and FIG. 8C is a side view of a non-woven three-dimensional implant.

FIG. 9A is a diagram of non-woven supports using the Mesh3 design.

FIG. 9B is a diagram of non-woven disks using the Mesh3 design.

FIG. 9C is a diagram of a unit cell of a non-woven soft tissue implant designated Mesh3

FIG. 9D is a display of various measured parameters within Mesh3 and the equations used to calculate the surface area ratio and surface area to volume ratio.

### DETAILED DESCRIPTION

The present invention features methods of making and using three-dimensional biocompatible implants, as well as the implants per se. An implant, or a subassembly (or collections of subassemblies) therein (e.g., a weft knit subassembly or warp knit subassembly), can be constructed to resist compression when implanted in a warm-blooded animal (e.g., a mammal such as a human) for a period of time (e.g., one to six months, a year, or more).

Weft knit subassemblies can include knit materials that are produced by machine or hand knitting with the fibers running crosswise or in a circle. Warp knit subassemblies can include knit materials that are produced by machine or hand knitting with the yarns running in a lengthwise direction. Either or both subassemblies can be used in the three-dimensional implants of the present invention. As illustrated in the figures below, the subassembly can include woven or braided fibers (including those conforming to the weft knit or warp knit patterns just described) of a biocompatible (i.e., not toxic) material such as a non-absorbable polymer (e.g., polypropylene, polyethylene terephthalate, polytetrafluoroethylene, polyaryletherketone, nylon, fluorinated ethylene propylene, polybutester, silicone, and the like), a nonwoven material of a biocompatible (i.e., not toxic) material such as a non-absorbable polymer (e.g., polypropylene, polyethylene terephthalate, polytetrafluoroethylene, polyaryletherketone, nylon, fluorinated ethylene propylene, polybutester, silicone, and the like), an absorbable polymer (polyglycolic acid, polylactic acid, polycaprolactone, polyhydroxyalkanoate, polyglyconate, or copolymers thereof (e.g., a PGA:PLA copolymer (the ratio of PGA to PLA can be about 50:50)), a metal (e.g., stainless steel or nitinol), or a tissue-based material (e.g., collagen or a collagen-based or collagen-containing material).

Any of the implants described herein can have an internal support material (e.g., an intraluminal support), such as a polymer (e.g., polypropylene, polyethylene terephthalate, polytetrafluoroethylene, nylon, fluorinated ethylene propylene, silicone, polyurethane, rubber) or a metal (e.g., nitinol). The implant can also include polyaryletherketone (PEEK). PEEK polymer has properties that make it useful in implants of the invention that will be used in or around tissues other than soft tissues. For example, a three-dimensional implant of the invention that includes an internal support material such as PEEK can be used in spine cages, bone screws, orthopedic stems, and dental implants (of course, PEEK-containing implants can be made and used to improve defects in soft tissues as well). Invibio Inc., Lancashire, UK, manufactures PEEK. PEEK offers a desirable combination of strength, stiffness, and toughness, together with extensive biocompatibility. Because the PEEK polymer has enhanced mechanical properties, it is well suited for low material content implants. Soft tissue implants can be fabricated from smaller diameter fibers or thin films with lower profiles than commercially available implants.

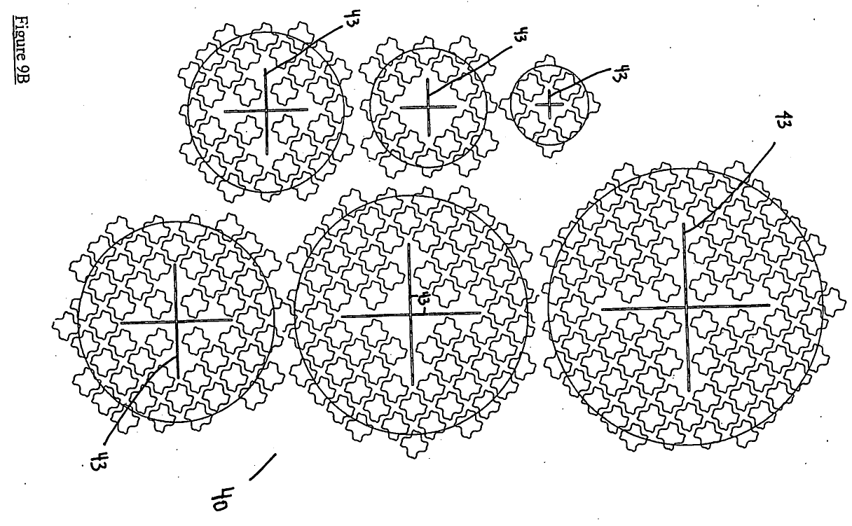

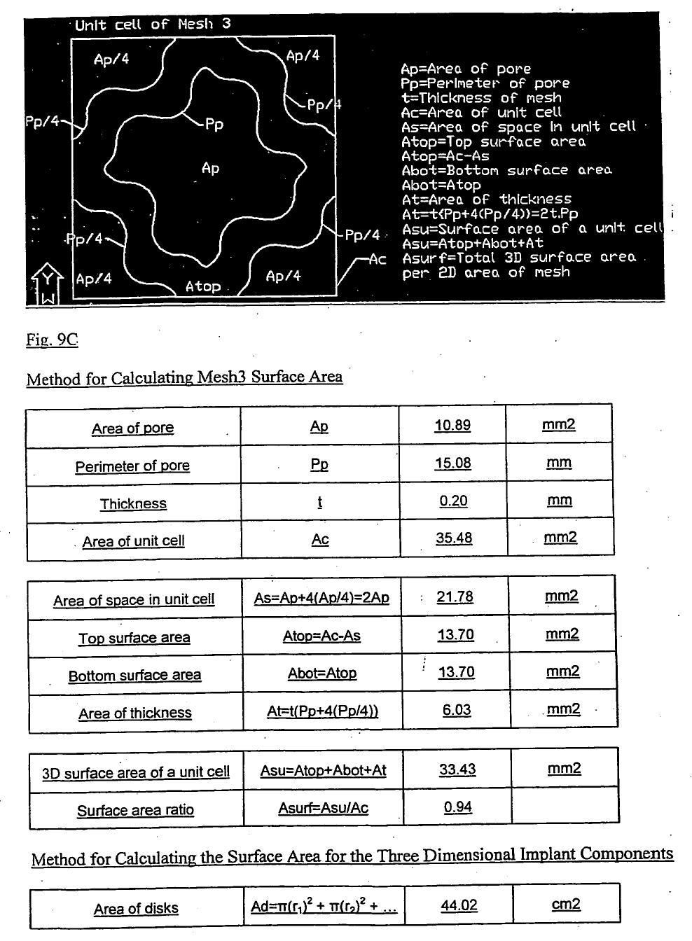

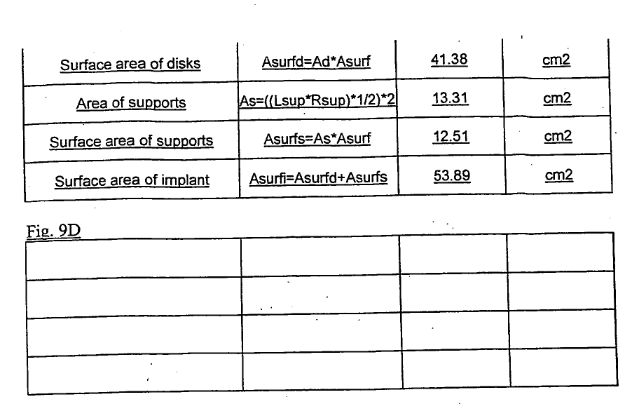

The implants (e.g., the subassemblies) can be produced using a circular weft knitting process (with or without an internal support (e.g., with or without an underlying polymer, as described above, in all or a portion of the subassembly)) or a circular warp knitting process. Alternatively, the implants (or subassemblies) can be produced using a braiding process. Alternatively, the implants (or subassemblies) can be produced using a porous biocompatible film with cell patterns having thickness of less than about 0.025 inches (an exemplary cell pattern is shown in FIG. 9C).

The implants can be produced by methods that include one or more of the following steps: extruding a biocompatible polymer into a fiber or film; transforming the fiber or film into a compression resistant subassembly; shaping, braiding, or weaving the subassembly into a three dimensional structure; heat setting the structure into the desired shaped article; and, optionally, attaching the shaped article to a complementary implant article (e.g., an anchor). These steps can be performed in the order given. The methods can also include removing shaping mandrels, internal supports, or intraluminal supports (where such are used, at, for example, the completion of the shaping, braiding, or weaving process).

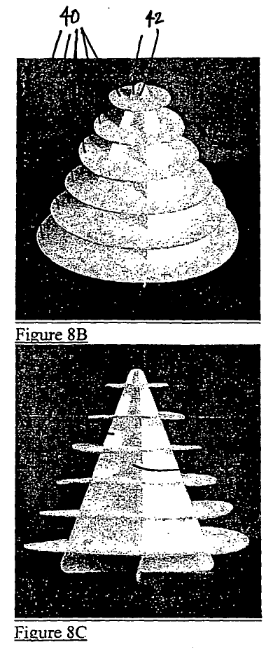

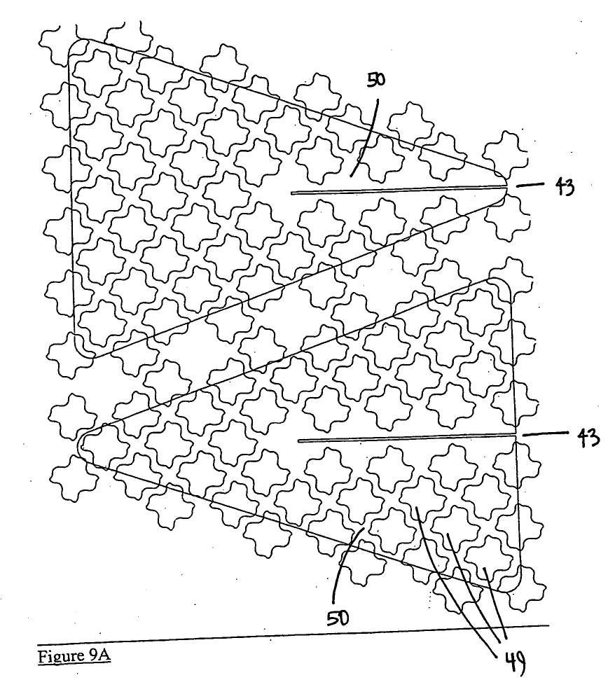

As noted elsewhere, the subassemblies can include pores of, for example, 50-2000 microns in diameter (when the implant is placed in a resting or non-compressed position). The implant can assume any number of forms, which may be tailored for use in particular parts of the body or in response to certain defects. For example, the implant can have a conical form (as shown, for example, in FIGS. 8A, 8B, and 8C).

The implant subassembly can have a surface area ratio less than 3.0 (e.g., 0.50-3.0 or, for example, about 1.0), and the implant (or one or more of the subassemblies therein) can include an additional component such as an onlay or anchor or other means for stabilizing the implant during placement within a warn-blooded animal. The implants (or one or more of the subassemblies therein) can be connected to one or more implant components (e.g., an onlay and/or anchor) in a manner that permits independent placement and stabilization of the implants.

The methods of the invention (the methods of generating a three-dimensional implant and the methods for implanting that implant into a patient) can be used to repair essentially any defective tissue. For example, a three-dimensional biocompatible implant described herein can be applied to a tissue defect by way of a surgical procedure (these procedures will be analogous to those carried out in the art using different types of implants). The patient being treated may have, for example, a hernia or other tissue rupture, tear, or defect. The methods can include exposing a defective tissue on or within the patient's body and placing the implant on or over the tissue; before or during placement, the implant can be compressed, by hand or by a device.

Biocompatible fibers (used in, for example, the subassemblies) can be produced using a melt extrusion process. Luxilon Industries NV (Wijnegem, Belgium) produces medical grade fiber suitable for this application. Luxilon produces polypropylene fiber used for implants. Lamb Knitting Machine Corporation (Chicopee, Mass.) produces knitting equipment suitable for this processing step. The fiber is converted using either a circular weft-knitting machine or a warp knit braider.

The weft or warp knit subassemblies, with or without an intraluminal support (e.g., a polymer support such as PEEK polymer), can be braided into a three-dimensional implant structure. For example, Wardwell Braiding Machine Company located in Central Falls, R.I. produces braiding equipment suitable for this processing step.

The braided three-dimensional implant structure can be heat set into a more stable structure by heating the three-dimensional implant structure above its glass transition temperature. A suitable temperature for polypropylene materials is 150° C. Mandrels can be used to support the subassemblies so that a desired shape with predetermined dimensions is produced.

Biocompatible films (used in, for example, the subassemblies) can be produced using an extrusion and orientation process. Bard Peripheral Vascular (Tempe, Ariz.) produces expanded polytetrafluoroethylene film suitable for this application. The film can be machined into a design with cell patterns to impart a higher degree of porosity with a lower implant surface area ratio. The film can be converted into a three-dimensional object (e.g., a cylinder, cone, sphere, or block (e.g., an essentially square or rectangular block) using a cutting and heat setting process.

Medical implant applications for the soft tissue implant technology described above may include, but are not limited to, plastic reconstruction, hernia repair, vessel occlusion and other soft tissue reconstruction procedures where biocompatible fillers are required. The soft tissue implant can be produced in a variety of shapes and sizes for the particular indications. The shaped article can also be used for blood filtration applications. Non-medical applications may include diagnostic, biotechnology, automotive, electronics, aerospace, and home and commercial appliances applications.

Referring now to the figures:

FIG. 1 is a perspective view of weft knit subassembly 14. The weft knit subassembly is made from biocompatible fiber 16 and has a known design and fiber count. Fiber count is characterized through needle and stitch densities for the material. The weft knit subassembly 14 is made of a biocompatible material.

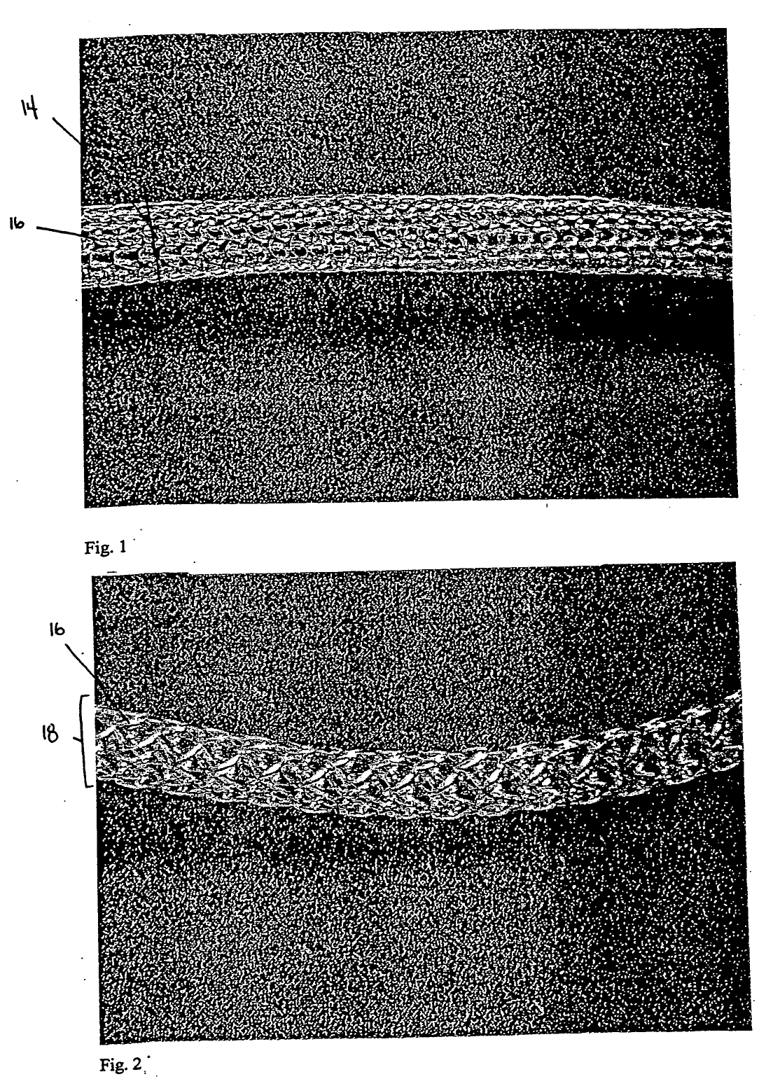

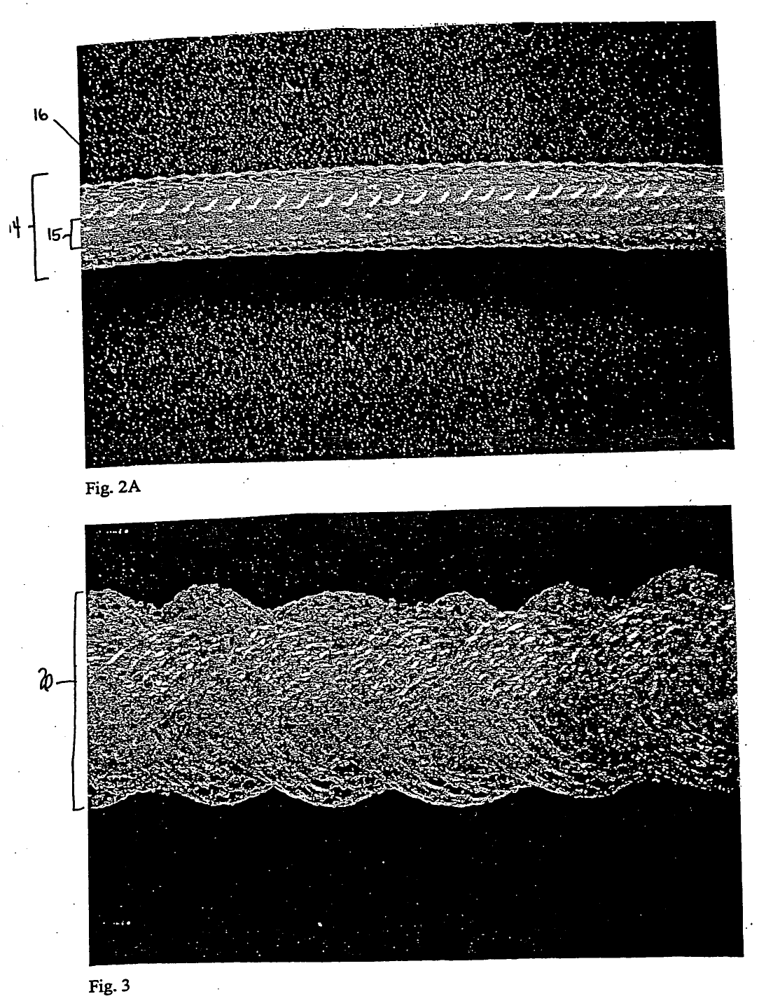

FIG. 2 is a perspective view of a warp knit subassembly 18. The warp knit subassembly 18 is made from biocompatible fiber 16 and has a known design and fiber count. The warp knit subassembly 18 is made of a biocompatible material.

FIG. 2A is a perspective view of a weft knit subassembly 14 with intraluminal support 15. Biocompatible fibers 16 are found external to and, optionally, in physical connection with intraluminal support 15. Intraluminal support 15 can provide a compression resistant structure during processing, and can be composed of a material that permits post-processing.

FIG. 3 is a perspective view of three-dimensional implant 20. Weft knit subassembly 14 has been converted into braided three-dimensional implant 20 using braiding equipment.

FIG. 4 is a perspective view of shaped three-dimensional implant 22 (a conical implant). The braided three-dimensional structure has been heat set into a shaped three-dimensional implant 22 by placing shaping mandrels in the weft knit subassemblies 14 (composed of biocompatiable fiber 16) and applying heat.

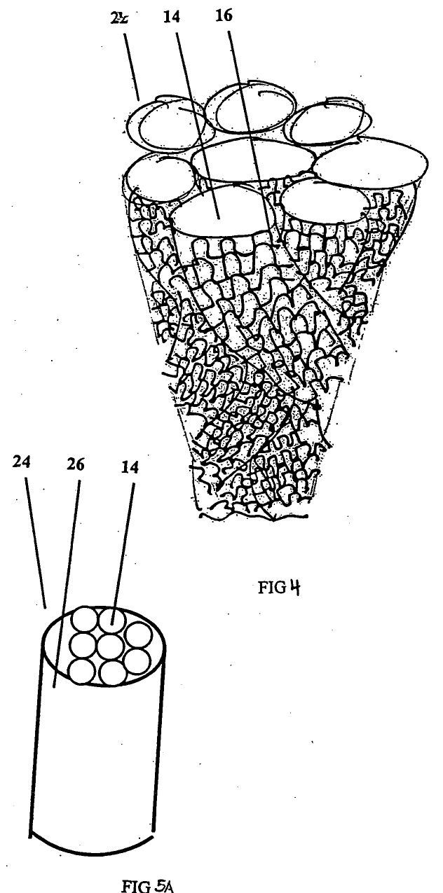

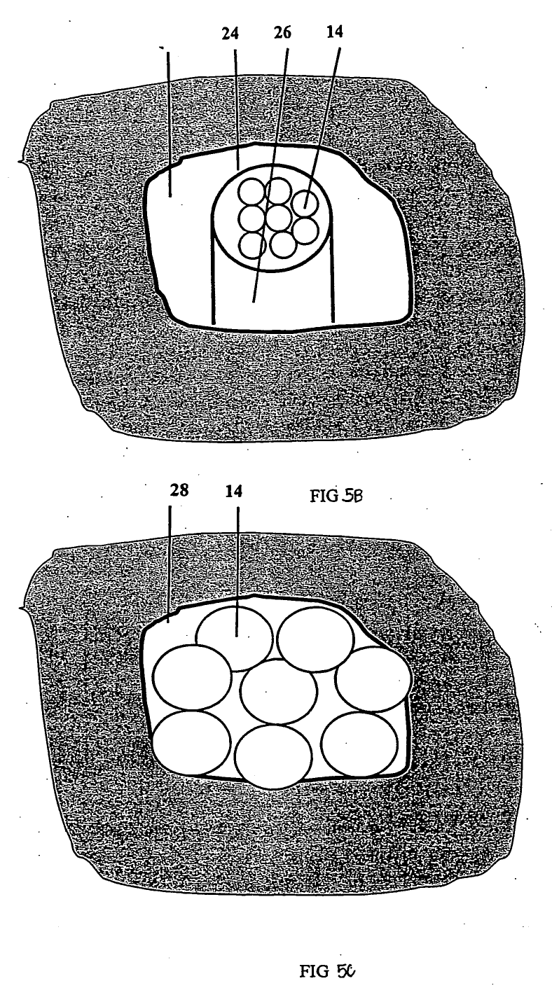

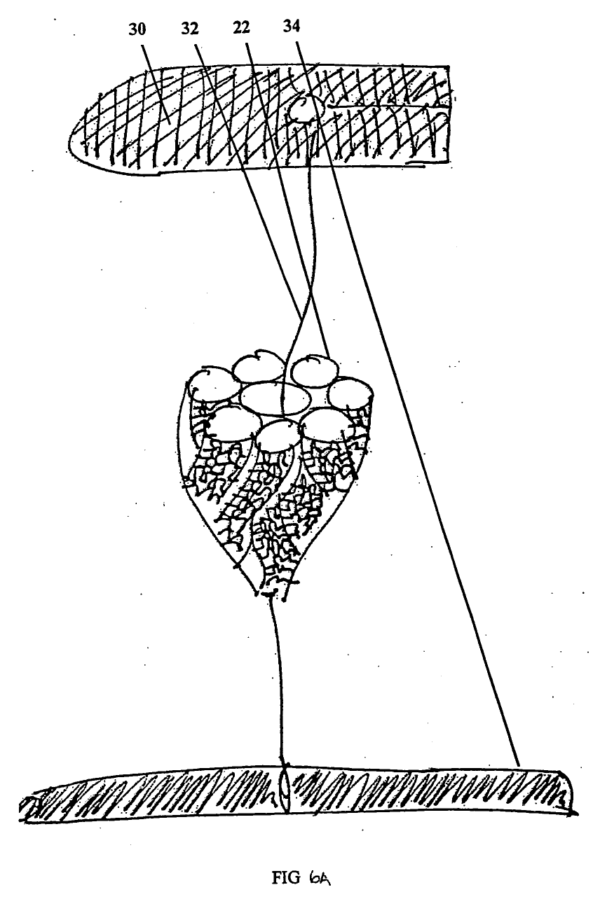

FIG. 5A is a perspective view of a constrained three-dimensional implant 24. The implant is collapsed and constrained by a hollow tube 26 to prevent expansion of the weft knit subassemblies 14.

FIG. 5B is a perspective view of a constrained three-dimensional implant 24 positioned in a bodily defect 28.

FIG. 5C is a perspective view of a shaped three-dimensional implant 22 unconstrained and filling the bodily defect 28.

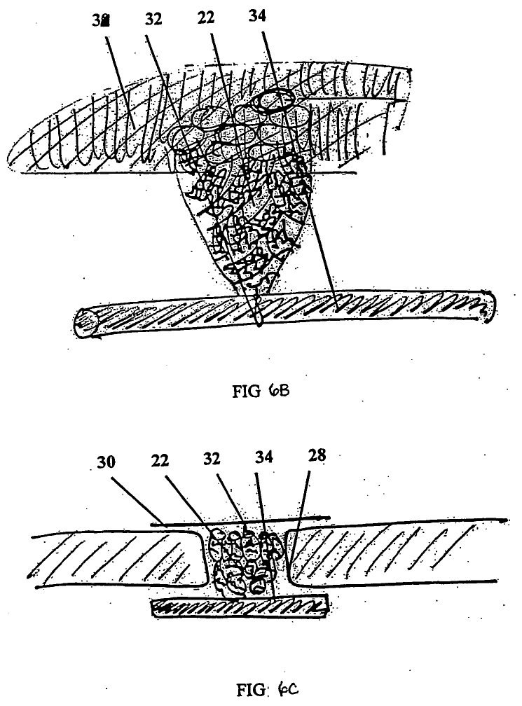

FIG. 6A is a perspective view of a shaped three-dimensional implant 22, implant onlay 30, and anchor 34 connected together with connecting filament 32 that permits individual placement of separate structures.

FIG. 6B is a perspective view of a shaped three-dimensional implant 22, implant onlay 30, and anchor 34 connected together with connecting filament 32 that prevents migration of the individual components.

FIG. 6C is a cross sectional view of a shaped three-dimensional implant 22, implant onlay 30, and anchor 34 connected together with connecting filament 32 positioned in a bodily defect 28.

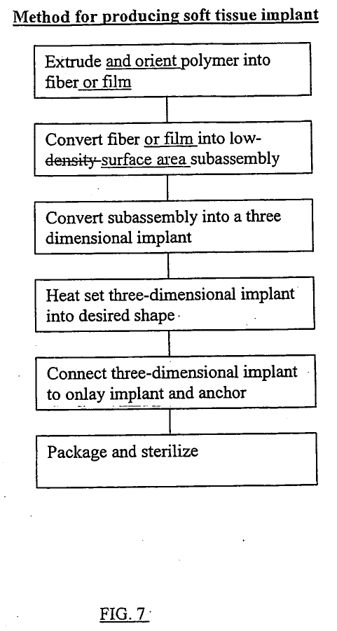

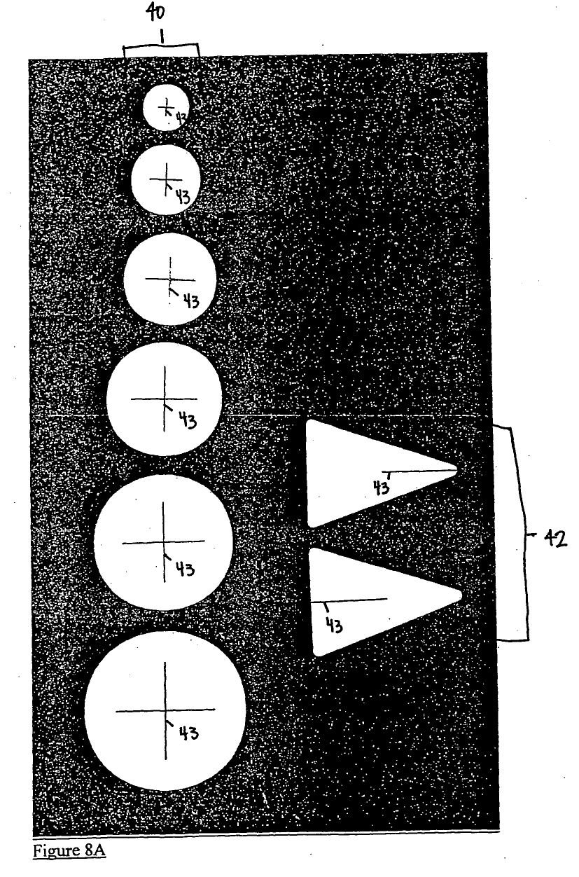

FIG. 7 is a diagram showing one possible combination of manufacturing steps.

FIG. 8A is a perspective view of a non-woven subassembly with biocompatible disks 40 and support members 42. Both disks 40 and support members 42 have openings 43, which allow the subassembly to slide together.

FIG. 8B is a perspective view of a non-woven three-dimensional implant with biocompatible disks 40 and support members 42.

FIG. 8C is a side view of a non-woven three-dimensional implant with biocompatible disks 40 and support members 42.

FIG. 9A is a diagram of non-woven supports 50 machined using the Mesh3 design with openings 43 to permit assembly. Pores 49

FIG. 9B is a diagram of non-woven disks 40 using the Mesh3 design with openings 43 which accommodate the support members.

FIG. 9C relates to a non-woven soft tissue implant designated Mesh3.

FIG. 9D is a display of various measured parameters within Mesh3 and the equations used to calculate the surface area ratio and surface area to volume ratio.

### EXAMPLE

### Example 1

A three-dimensional non-woven soft tissue implant was constructed using a biaxially-oriented polymer film. The film is stretched in both the machine and transverse directions (relative to the extrusion direction) to orient the polymer chains. The stretching process can take place simultaneously or sequentially depending on the equipment that is available. The base film was Syncarta™ (AET Films, Peabody, Mass.). The base film was machined into Mesh Design 3 (“Mesh3”) using a 3.0-Watt Avia Q-switched Ultraviolet Laser produced by Coherent, Inc. (Santa Clara, Calif.). The design of a cell for the non-woven soft tissue implant is shown in FIG. 9C. The soft tissue implant was cut into circular disks and triangular supports used to construct a three-dimensional implant. The calculation for the surface area for the components used to construct the three-dimensional implant is shown in FIG. 9D.

Vᵢₘₚₗₐₙₜ=((II)(Lᵢₘₚₗₐₙₜ)(Rᵢₘₚₗₐₙₜ)²)/3 where Vᵢₘₚₗₐₙₜ is the volume of the cone shaped implant, Lᵢₘₚₗₐₙₜ is the implant height, and Rᵢₘₚₗₐₙₜ is the implant radius at the base; and

Surface Area to Volume Ratio A_{surface implant}/Vᵢₘₚₗₐₙₜ.

| | Implant | Implant | Surface |
|---|---|---|---|
| | Surface Area | Volume | Area to |
| Product | (cm²) | (cm³) | Volume Ratio |
| Three Dimensional | 53.89 | 21.61 | 2.49 |
| Implant (Mesh Design 3) | | | |

Although the description above contains many specificities, these should not be construed as limiting the scope of the invention but as merely providing illustrations of some of the presently preferred embodiments of this invention. For example, the implant can have other subassembly designs, different materials can be utilized, and alternate equipment can be used to produce the structures, etc.

What is claimed is:

1. A three-dimensional biocompatible implant, the implant comprising a subassembly that resists compression when implanted in a warm-blooded animal.

2. The implant of claim 1, wherein the subassembly comprises woven or braided fibers.

3. The implant of claim 1, wherein the subassembly is produced using a circular weft knitting process.

4. The implant of claim 3, wherein the subassembly is produced using a circular weft knitting process with an internal support.

5. The implant of claim 3, wherein the subassembly is produced using a circular weft knitting process without an internal support.

6. The implant of claim 1, wherein the subassembly is produced using a circular warp knitting process.

7. The implant of claim 1, wherein the subassembly is produced using a braiding process.

8. The implant of claim 1, wherein the subassembly is produced using a nonwoven film and/or wherein the subassembly comprises pores.

9. The implant of claim 8, wherein the pores are 50-2000 microns in diameter.

10. The implant of claim 9, wherein the implant has a conical form.

11. The implant of claim 1, wherein the implant comprises polyaryletherketone.

12. The implant of claim 1, further comprising an onlay

13. The implant of claim 1, further comprising an anchor.

14. The implant of claim 1, further comprising a means for stabilizing the implant during placement within a warm-blooded animal.

15. A method for producing a three-dimensional biocompatible implant, the method comprising one or more of the following steps:
a) extruding a biocompatible polymer into a fiber,
b) transforming the fiber into a compression resistant subassembly,
c) braiding or weaving the subassembly into a three dimensional structure,
d) heat setting the structure into the desired shaped article, and, optionally,
e) attaching the shaped article to a complementary implant article.

16. The method of claim 15, further comprising removing shaping mandrels or intraluminal support.

17. A method for repairing a defective tissue in a patient, the method comprising applying the three-dimensional biocompatible implant to the defect by way of a surgical procedure.

18. The method of claim 17, wherein the patient has a hernia.

19. A kit comprising an implant of claim 1, wherein the implant is sterile.

20. A method of delivering the implant of claim 1 to a patient's body, the method comprising exposing a defective tissue on or within the patient's body and placing the implant on or over the tissue.

21. The method of claim 20, wherein the implant is compressed, by hand or by a device, prior to being placed on or over the tissue.

22. A method for producing a three-dimensional biocompatible implant, the method comprising one or more of the following steps:
a) extruding a biocompatible polymer into a film,
b) transforming the film into a subassembly,
c) shaping the subassembly into a three dimensional structure,
d) heat setting the structure into the desired shaped article, and, optionally,
e) attaching the shaped article to a complementary implant article.

23. The implant of claim 1, wherein the implant has a surface area to volume ratio less than about 5.0.

24. The three dimensional implant of claim 23, wherein the surface area to volume ratio is less than about 4.0, less than about 3.0, less than about 2.0, or is about 1.0.

25. The three-dimensional implant of claim 23, wherein the biocompatible material comprises a non-absorbable polymer or copolymer.

26. The three-dimensional implant of claim 25, wherein the non-absorbable polymer or copolymer comprises polypropylene, polyethylene terephthalate, polytetrafluoroethylene, polyaryletherketone, nylon, fluorinated ethylene propylene, polybutester, or silicone.

27. The three-dimensional implant of claim 23, wherein the biocompatible material comprises an absorbable polymer or copolymer.

28. The three-dimensional implant of claim 27, wherein the absorbable polymer or copolymer comprises polyglycolic acid (PGA), polylactic acid (PLA), polycaprolactone, or polyhydroxylkanoate.

29. The three-dimensional implant of claim 23, wherein the biocompatible material comprises a biological material.

30. The three-dimensional implant of claim 29, wherein the biocompatible material is collagen.

31. A three-dimensional implant comprising two or more layers of two-dimensional biocompatible material with interconnecting supports, said implant constructed to securely fit within a tissue or muscle wall defect.
